(19) United States
(12) Patent Application Publication
(10) Pub. No.: US 2007/0027202 A1
(43) Pub. Date: Feb. 1, 2007

(54) Process for the preparation of carvedilol and its salts

(76) Inventors: Ashok Kumar, Mumbai (IN); Ashvini Saxena, Ratlam (IN); Anindya Battacharyya, Ratlam (IN); Amit Vikram Singh Sengar, Ratlam (IN); Gunjan Pramod Pathak, Ratlam (IN); Satish Rajanikant Soudagar, Mumbai (IN); Pramil Kumar Mathur, Mumbai (IN); Avinash Manohar Nijasure, Mumbai (IN); Sanjukumar Motiram Salunke, Mumbai (IN); Prashant Gautam, Mumbai (IN); Thakur Gajendrasingh Ramsingh, Mumbai (IN); Dilip Uttam Jadhav, Mumbai (IN)

Correspondence Address: BLANK ROME LLP, 600 NEW HAMPSHIRE AVENUE, N.W., WASHINGTON, DC, 20037, US

(21) Appl. No.: 11/480,526

(22) Filed: Jul. 5, 2006

(30) Foreign Application Priority Data
Jun. 7, 2005 (IN) 807/MUM/2005

(51) Int. Cl. C07D 209/82; A61K 31/403

(52) U.S. Cl. 514/411; 548/444

(57) ABSTRACT

Disclosed herein is a process for preparation of carvedilol substantially free from its bis-impurity comprises the reaction of 4-(2,3-epoxypropoxy)carbazole and 2-(2-methoxyphenoxy)ethylamine in a polar aprotic solvent media; followed by isolation of carvediol from the reaction mass as an acid addition salt and subsequent conversion into pure carvedilol.

### FIELD OF INVENTION

The present invention relates to a preparation method for 1-(9H-Carbazol-4-yloxy)-3-[[2-(2-methoxy phenoxy)ethyl]amino]-2-propanol and its salts in higher purity.

### BACKGROUND OF THE INVENTION

The pharmaceutically valuable compound 1-(9H-Carbazol-4-yloxy)-3-[[2-(2-methoxy phenoxy)ethyl]amino]-2-propanol, also known as carvedilol under the international proprietary name, is a non-selective β-adrenergic blocking agent with vasodilating activity. Carvedilol is represented with the following structural formula:

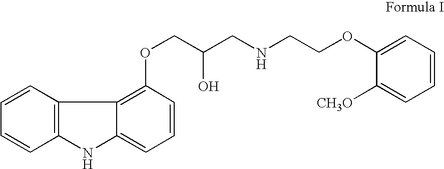

Carvedilol has one chiral carbon atom and hence it can exist in either as its stereoisomers or in its racemic form.

Carvedilol was reported in EP 000 4920, wherein the compound 4-(2,3-epoxy propoxy) carbazole of Formula II is reacted with 2-(2-methoxy-phenoxy)ethylamine of Formula III to prepare the carvedilol as per the reaction sequence shown in Scheme I:

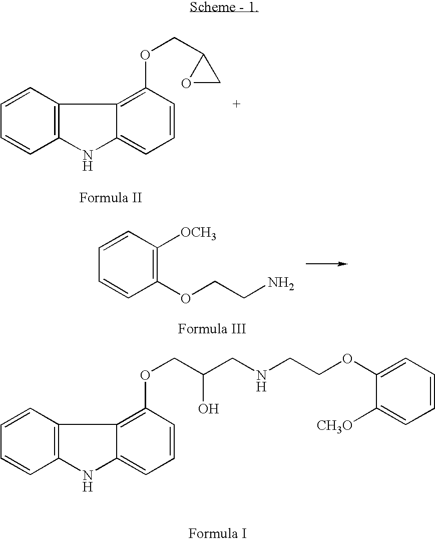

The known processes produce carvedilol in lower yield with formation of a bis impurity of Formula IV, which in most cases cannot be avoided, in about 10-15% of the total product. This necessitates additional purification procedures for the isolation of carvedilol free from contamination of high percentage of bis-impurity and therefore the process may not be ideal for industrialization.

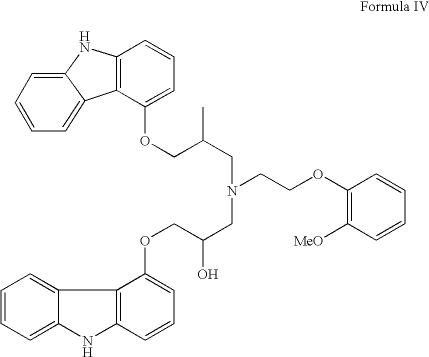

EP 0918 055 discloses a process by which the formation of bis impurity is avoided (Scheme II) by reacting a benzyl protected 2-(2-methoxy phenoxy)ethylamine (Formula V) with 4-(2,3-epoxy propoxy)carbazole. The intermediate benzyl carvedilol (Formula VI) has to be debenzylated before isolating the product carvedilol. This process introduces two new steps namely, benzyl protection of 2-(2-methoxy phenoxy) ethylamine and deprotection of the benzyl carvedilol obtained after the condensation reaction. In addition, the process involves debenzylation of the protected moiety by employing either costly metal catalyst or hydrazine hydrate in the synthesis of carvedilol affecting the economy of the process.

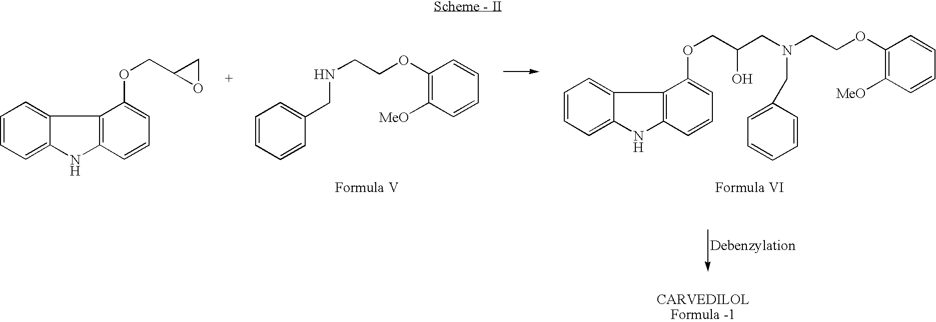

WO 01/87837 describes the preparation of carvedilol from 4-hydroxy carbazol by reacting it with 5-chloromethyl-3-[2-(2-methoxy phenoxy)ethyl]-oxazolidin-2-one (Formula VII) to yield oxazolidine derivative of Formula VIII as expressed in Scheme III. This intermediate is further hydrolyzed to get carvedilol. The preparative process for the intermediate of Formula VII involves various stages, such as reacting 1,3-dichloropropan-2-ol with phenyl chloroformate, which is made to react with 2-[2-(methoxy phenoxy)]ethyl amine hydrochloride to yield [2-2-methoxy phenoxy)-ethyl]carbamic acid-2-chloro-1-chloromethyl-ethyl ester. The ester formed is cyclised to get compounds of Formula VII. In order to produce carvedilol, free of bis impurity, the process introduces number of additional steps making it cumbersome for the industrial production of the compound.

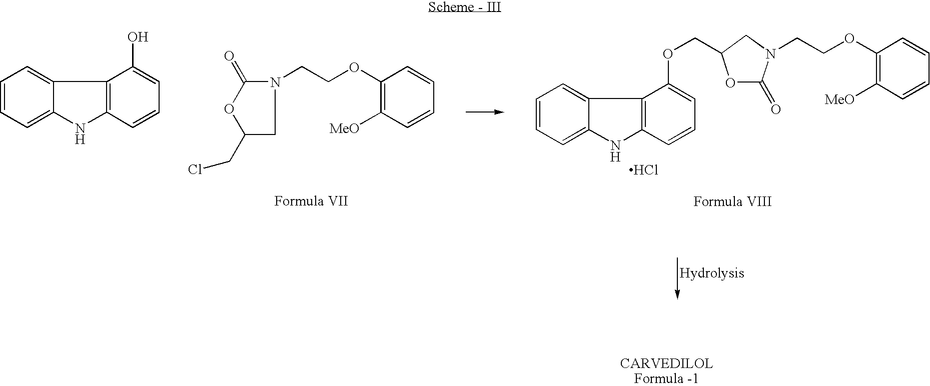

WO 02/00216 describes the preparation of carvedilol from 4-(2,3-epoxy propoxy) carbazole (Formula II) with 2-(2-methoxy phenoxy)ethylamine (Formula III). The process uses a large molar excess of intermediate of Formula III in the range of 2.80 moles to 100 moles per 1.0 mole of 4-(2,3-epoxy propoxy)carbazole (Formula II) to avoid the formation of bis impurity (Formula IV). The application further discloses the process for isolation of carvedilol as crystalline Hydrochloride, Hydrochloride Hydrate, and methyl ethyl ketone solvate. The use of large excess of 2-(2-methoxy phenoxy) ethylamine (Formula III) makes the process uneconomical and requires the excess reactant to be recovered and recycled to make the process economically successful.

Further, WO 2004/094378 describes a process for preparation of carvedilol Form-II by reacting 4-(2,3-epoxy propoxy)carbazole (Formula II) with 2-(2-methoxy phenoxy) ethylamine (Formula III) in solvents like monochlorobenzene, monoglyme or mixtures thereof. The application does not mention about the bis impurity formed during the condensation stage.

There remains a long-standing need in the industry to provide an economical process for preparation of carvedilol with control over the formation of the bis impurity of Formula IV.

### SUMMARY OF THE PRESENT INVENTION

The main objective of the present invention is to provide an economical and industrially feasible process for the preparation of carvedilol.

Yet another objective of the present invention is to minimize the formation of bis impurity and isolate carvedilol free from contaminating bis-impurity of formula IV in high purity and yields.

The present inventors have developed a process where the problem of formation of the bis impurity is limited to acceptably low amount without using large excess of reactants or adding new reactants/reagents or introducing extra process steps in the condensation reaction of compounds of Formula II & III, which is the subject of the present invention.

Accordingly a process is provided for the preparation of carvedilol which comprises reacting 4-(2,3-epoxy propoxy)carbazole (Formula II) with 2-(2-methoxy phenoxy) ethylamine (Formula III) in a media of polar aprotic solvent selected from dimethyl sulfoxide, dimethyl acetamide, and N-methyl pyrollidone at a temperature ranging from about 50 to 100° C. to form a reaction mass containing carvedilol with minimum bis-impurity. The reaction in these selected solvents minimizes the formation of the bis-impurity to less than about 7%. The invention also provides an isolation procedure for obtaining carvedilol substantially free of its bis-impurity of Formula IV.

Thus, the present invention provides a process for purification of carvedilol containing the known bis impurity (Formula IV) by subjecting the crude carvedilol to either repeatedly leaching in toluene or suspending/dissolving the contaminated carvedilol into a water immiscible organic solvent, washing the organic phase with an aqueous acid till the pH of the washings is in the range of 7.0 to 8.0; further, adding an acid to organic layer to attain a pH of 4 to 5, thereby selectively precipitating the carvedilol as an acid salt, isolating carvedilol after an acid-base treatment, which is further leached in toluene, water or mixture thereof; and finally crystallizing the carvedilol from ethyl acetate to obtain carvedilol substantially free of bis-impurity.

### DETAILED DESCRIPTION OF THE INVENTION

The process of the present invention is described herein after in more details substantiating various embodiments and conditions of reaction for better understanding of the invention.

The process of the present invention describes the preparation of carvedilol of Formula I by reacting 4-(2,3-epoxy propoxy)carbazole (Formula II) with 2-(2-methoxy phenoxy)ethylamine (Formula III) in presence of selected polar aprotic solvent. The reaction carried out in polar aprotic solvent has found to limit the formation of the bis-impurity to about 5-7% in comparison with 10-20% in reported solvents like alcohols, acetonitrile, ethoxyethanol, hydrocarbon solvents etc. or in neat reaction.

The polar aprotic solvent suitable for the reaction is selected form dimethyl sulfoxide, dimethyl acetamide, and N-methyl pyrollidone, and the most preferred solvent for the condensation reaction is dimethyl sulfoxide. The reaction is preferably performed by heating the reactants in the said solvent medium and the preferred temperature for carrying out the reaction is by maintaining the reaction mass at 68-72° C. The reaction normally completes in a span of 15-20 hours. 2-(2-methoxy phenoxy)ethylamine reactant used in the reaction is preferably in slight excess in the range of 1.5 to 2.5 molar equivalents relative to the compound 4-(2,3-epoxy propoxy)carbazole.

In this process, the work up and isolation of the product formed is carried out either by quenching the mass directly in water or the solvent is first distilled and then it is quenched in water and extracted the free base in organic solvent like dichloro methane. The preferred method of work up is to quench the reaction mixture in water without distilling the solvent and extracting the solution with solvent dichloro methane.

Further, the carvedilol reaction mixture is purified from excess reactant and bis-impurity by the following procedure. The organic solvent extract obtained above is washed one or more times till the pH of the last washings is reached to 7.0-7.5 with the help of an aqueous acid solution, and the aqueous layer is separated off. The adjustment of pH to 7.0-7.5 and separation of aqueous layer is necessary to remove the contamination of 2-(2-methoxy phenoxy)ethylamine (Formula-III) and other impurities. The aqueous acid used for the pH adjustment is selected from hydrochloric acid, sulphuric acid, hydrobromic acid, phosphoric acid, p-toluene sulphonic acid and acetic acid. After removing the aqueous layer, the pH of the organic layer is adjusted to 4 to 4.5 using an acid, preferably with the same acid to selectively precipitate carvedilol as an acid salt. The preferred acid for the pH adjustment is dilute hydrochloric acid, sulphuric acid, p-toluene sulphonic acid and acetic acid. The acids used to form salt of carvedilol include the above described acids as well as sulphonic acid or dibasic organic acids. The sulphonic acid is selected form methane sulphonic acid, p-toluene sulphonic acid and benzene sulphonic acid. The dibasic acids include succinic acid, phthalic acid, maleic acid, malonic acid etc. Most of the bis-impurity is removed by forming the salt of carvedilol in this isolation step. The precipitated carvedilol salt is then subjected to an acid-base treatment in an organic solvent for the isolation of crude carvedilol free base. The base used for liberating carvedilol from the corresponding acid salt is selected from aqueous ammonia solution, sodium bi carbonate, sodium carbonate, and triethyl amine.

The crude carvedilol isolated is leached in solvent selected from toluene, water or its mixture thereof and stirred at 25-60° C. and filtered. The preferred solvent for leaching is a mixture of toluene and water. If necessary this leaching procedure is repeated to get carvedilol substantially free of bis-impurity.

The crude carvedilol is crystallized by dissolving in ethyl acetate at an elevated temperature of 76-80° C. to get a clear solution. The carvedilol solution is optionally treated with an adsorbent and filtered to remove the adsorbent, concentrate the solution to ⅓^{rd} of the volume and subsequently cooled to 20-30° C. to crystallize out the pure product. The preferable concentration of carvedilol relative to the solvent is in an amount of about 5 to 25 ml per gram of carvedilol and more preferably about 10 ml per gram of carvedilol.

The product obtained is filtered and dried to get pure carvedilol. The carvedilol obtained by the crystallization according to this procedure is almost free from bis impurity. The crystalline carvedilol so obtained has purity of more than 99.50% (By HPLC).

The following non-limiting examples presented to illustrate the best mode of carrying out the process of the present invention. The examples are not limited to the particular embodiments illustrated herein but include the permutations, which are obvious set forth in the description.

### EXAMPLE 1

### Preparation of PTSA Salt of Carvedilol

a) In a dry reaction flask, 120 gm (0.502 moles) of 4-(2,3-epoxypropoxy)carbazole of Formula II, 188.7 gm (1.13 mole) of 2-(2-methoxy phenoxy)ethylamine, and 1200 ml dimethylsulphoxide (DMSO) were charged under dry nitrogen atmosphere. The reaction mass was heated to about 70° C. till completion of reaction (about 20 hours), and then the reaction mass was cooled to 30° C. and 1200 ml water was added to it. The crude product was extracted with dichloromethane (1200 ml), and the dichloromethane layer was washed with water. The dichloromethane layer was mixed with 240 ml water, followed by the addition of 55.8 gm p-toluene sulphonic acid (PTSA) to attain a pH in the range of 7 to 8. After stirring, the layers were separated and the organic layer was washed with water.
b) Above organic layer was taken in a reaction flask and to it PTSA (about 130 gm) was added to get a pH of the reaction mass about 4-5. After stirring, the PTSA salt of carvedilol was filtered and washed with dichloromethane. The wet cake is dried to get 215 gm of carvedilol.PTSA salt.

### EXAMPLE 2

### Preparation of Crude Carvedilol

190 gm of Carvedilol PTSA salt was taken in a flask, which was mixed with 1600 ml ethyl acetate, and 10% aqueous sodium carbonate solution (about 950 ml) till the pH is basic. The ethyl acetate layer was separated and distilled under vacuum to obtain a residue. 250 ml Toluene was added to the resulting residue after distillation of ethyl acetate and the precipitate obtained was filtered and dried to obtain 118 gm crude Carvedilol (88% yield).

### EXAMPLE 3

### Purification of Carvedilol

110 gm of crude carvedilol was dissolved in about 1300 ml of ethyl acetate at elevated temperature of 76 to 80° C. to obtain a clear solution and optionally the clear solution was treated with charcoal and filtered to remove the insoluble. The ethyl acetate solution was concentrated to about ⅓^{rd} and cooled the solution to a temperature of 20-30° C. to precipitate pure carvedilol. The obtained crystals were filtered off and dried to obtain 88 gm of pure carvedilol (80% yield).

### EXAMPLE 4

### Preparation of Carvedilol

In a dry reaction flask charged 25.0 g 4-(2,3-epoxy propoxy)carbazole (0.104 moles), 39.35 g of 2-(2-methoxy phenoxy)ethylamine (0.235 moles) in 250 ml dimethyl sulfoxide. The temperature of the reaction mass was raised to about 70° C. under stirring and maintaining the reaction mixture at 68-72° C. for 18-20 hrs. The reaction mass was cooled to about 30° C. and quenched the reaction mass in 250 ml water, stirred and extracted the resultant solution in 250 ml dichloromethane. The organic layer was separated and washed with aqueous sulphuric acid till pH of the washings about 7.0-8.0. The organic layer was separated and further adjusted the pH with the aqueous sulfuric acid to 4.0-4.5 to precipitate carvedilol sulphate salt. The precipitated salt was filtered and taken in 300 ml ethyl acetate and made alkaline with 10% sodium carbonate solution. The reaction mass was stirred and separated the organic layer. The ethyl acetate was distilled under vacuum, and 330 ml toluene was added to it. The solid obtained was filtered and crystallized from ethyl acetate to obtain pure carvedilol (58-62% yield).

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A process for making carvedilol (Formula I) 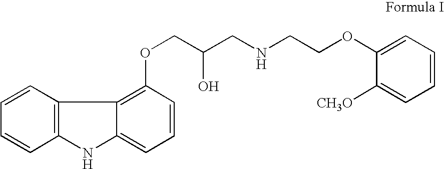
comprising the steps of
a. reacting the 4-(2,3-epoxypropoxy)carbazole (Formula II) and 2-2-methoxyphenoxy)ethylamine (Formula III) 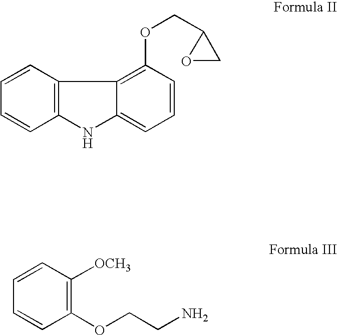
in a polar aprotic solvent media selected from dimethyl sulfoxide, dimethyl acetamide, and N-methyl pyrollidone; and
b. recovering carvedilol substantially free of 1,1-[{2-(2-methoxyphenoxy)ethyl}nitrilo]bis[3-(9H-carbazol-4-yloxy)propan-2-ol] impurity (Formula IV)

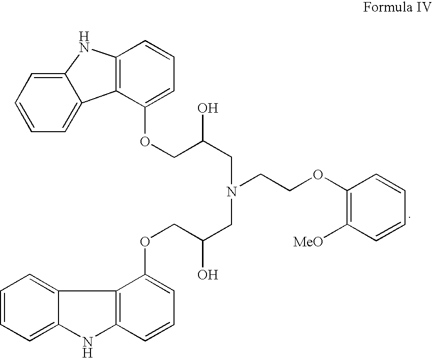

2. The process as claimed in claim 1 wherein the recovery of carvedilol further comprises:
a. forming a solution of crude carvedilol in an organic solvent;
b. washing said solution with an aqueous acid till the pH of the washings in the range of about 7.0-8.0;
c. adjusting pH of the carvedilol reaction solution to a pH greater than 4.0 with an acid to selectively form a carvedilol acid salt substantially free of the impurity of Formula IV; and
d. transforming carvedilol acid salt into carvedilol base substantially free of the impurity of Formula IV.

3. The process as claimed in claim 2, wherein the organic solvent is a chlorinated hydrocarbon such as dichloromethane or ethylene chloride.

4. The process as claimed in claim 2, wherein said acid is selected from hydrochloric acid, sulphuric acid, p-toluene sulphonic acid, acetic acid and phosphoric acid.

5. The process as claimed in claim 2, wherein said carvedilol salt is selected from the group consisting of sulphate, p-toluene sulphonate, acetate, and phosphate.

6. The process as claimed in claim 7, wherein the carvedilol salt is p-toluene sulphonate salt.

7. The process as claimed in claim 1, wherein the recovery of carvedilol comprises subjecting the crude carvedilol to repeated leaching by toluene or water or their mixture thereof.

8. The process as claimed in claim 1, wherein the carvedilol is further purified by crystallization from ethyl acetate.

9. The process as claimed in claim 1, wherein the starting 2-(2-methoxy phenoxy) ethylamine is used in molar excess relative to 4-(2,3-epoxy propoxy)carbazole.

10. The process as claimed in claim 1, wherein the molar ratio of 2-(2-methoxy phenoxy)ethylamine is in the range of 1.5 to 2.5 relative to the 4-(2,3-epoxy propoxy)carbazole employed.

11. A pharmaceutical composition comprising carvedilol substantially free from the impurity of Formula IV, wherein the carvedilol is obtained according to claim 1.
